(19) United States
(12) Patent Application Publication
(10) Pub. No.: US 2004/0076673 A1
(43) Pub. Date: Apr. 22, 2004

(54) Oral pharmaceutical composition containing a block copolymer

(76) Inventors: Nicola Bateman, Northwich Cheshire (GB); Julie Cahill, Lymm Cheshire (GB)

Correspondence Address: ROPES & GRAY LLP, ONE INTERNATIONAL PLACE, BOSTON, MA, 02110-2624, US

(21) Appl. No.: 10/415,677

(22) PCT Filed: Nov. 7, 2001
(86) PCT No.: PCT/SE01/02470

(30) Foreign Application Priority Data
Nov. 9, 2000 (GB) 0027375.5
Feb. 27, 2001 (GB) 0104751.3

(51) Int. Cl. A61K 9/14

(52) U.S. Cl. 424/486

(57) ABSTRACT

The invention relates to oral pharmaceutical compositions that comprise a water miscible micelle forming block copolymer and a compound. The copolymer can be a diblock copolymer of formula AB or BA. The copolymer could also be triblock copolymer of formula ABA or BAB, or a multiblock copolymer having repeating BA or AB units of formula A(BA)n or B(AB)n, where n is an integer. The A-block may be poly(L-lactide) or poly(D-, L-, or DL-lactic acid) and the B-block a polyethylene glycol.

The invention relates to oral pharmaceutical compositions which comprise a water miscible micelle forming block copolymer (hereinafter called “the copolymer”) and a compound. The copolymer can be a diblock copolymer of formula AB or BA. However the copolymer could also be a triblock copolymer of formula ABA or BAB. The copolymer could also be a multiblock copolymer having repeating BA or AB units of formula A(BA)n or B(AB)n, where n is an integer and wherein

A is selected from a group consisting of

poly D-, L-, DL-lactic acid,

poly D-, L-, DL-lactide,

poly-glycolic acid,

polyglycolide,

polylactide-co-glycolide,

poly-ε-caprolactone, and

poly(3-hydroxybutyric acid); and

B is selected from a group of hydrophilic polymers consisting of

polyvinylalcohol,

polyvinylpyrrolidone,

polyethylene oxide, and

polyethylene glycol; or the hydrophilic polymer B may itself be a copolymer, for example a

polyoxyethylene/polyoxypropylene block copolymer of the type known as Pluronics or synperonics.

Copolymers of the type described above are known, see for example U.S. Pat. Nos. 4,942,035, 745,160, 4,526,938 or EPO, 166,596, B1. Specifically these types of polymers are used in the formulation of parenteral compositions of drugs due to the ability of the copolymer to provide release of the drug over a prolonged period, several days. Previously it has not been thought that these polymers were suitable for oral administration due to the prolonged periods of release of drug, which would be unsuitable for achieving ideal oral adsorption of drug.

We have surprisingly found that such polymers are indeed suitable for oral administration of compounds and are particularly suitable for formulation to produce oral compositions of compounds with low aqueous solubility (less than 0.1 mg/ml at the site of absorption). Whilst not wishing to be bound by theory we believe that these coploymers act by a combination of dissolution enhancement and prevention of precipitation and thus can greatly increase levels of drug absorption after oral administration.

In particular the polymers are particularly good with compounds which have significantly lower solubility in the pH conditions encountered at the site of adsorption, typically the duodenum, ileum or colon, than in the stomach. Typically these are basic compounds which are more soluble in the acidic stomach than the more alkaline conditions found in the site of absorption.

Compounds which have low aqueous solubility or basic compounds may produce problems in their absorption possibly producing unacceptable levels of variability in absorption between patient and between dose.

A common factor which may affect the absorption of a drug when administered orally is the changing pH experienced by the drug as it passes through the GI tract. Typically a drug may be absorbed in any number of the following sites when administered orally; cheek lining, stomach, duodenum, ileum and colon. The pH maybe different at each site of adsorption with the pH significantly different from the stomach (pH 1-3.5) to the small intestine (pH 4-8). The solubility of the drug may vary with pH leading to the possibility of the drug coming out of solution as it passes through the GI tract. Particular difficulties exist where the drug is dissolved and the solubility decreases in the pH environment found at the site of adsorption. This leads to possible low absorption and variable adsorption between doses and different patients. For example we have found with the drug 1-(6-chloronaphth-2-ylsulfonyl)-4-[4-(4-pyridyl)benzoyl] piperazine (hereinafter referred to as Compound 1) is soluble within the acidic pH of the stomach, but is not adsorbed from this area, but has low solubility in the duodenum, ileum and colon which are the main sites of adsorption. Compound 1 possesses Factor Xa inhibitory activity at concentrations which do not inhibit, or which inhibit to a lesser extent, the enzyme thrombin which is also a member of the blood coagulation enzymatic cascade.

Compound 1 is disclosed as Example 3 of WO9957113.

Compound 1 possesses activity in the treatment or prevention of a variety of medical disorders where anticoagulant therapy is indicated, for example in the treatment or prevention of thrombotic conditions such as coronary artery and cerebro-vascular disease. Further examples of such medical disorders include various cardiovascular and cerebrovascular conditions such as myocardial infarction, the formation of atherosclerotic plaques, venous or arterial thrombosis, coagulation syndromes, vascular injury (including reocclusion and restenosis following angioplasty and coronary artery bypass surgery, thrombus formation after the application of blood vessel operative techniques or after general surgery such as hip replacement surgery, the introduction of artificial heart valves or on the recirculation of blood), cerebral infarction, cerebral thrombosis, stroke, cerebral embolism, pulmonary embolism, ischaemia and angina (including unstable angina).

Standard tablet formulations of compound 1 may not be satisfactory due to the above reasons and have lead to poor oral bioavailability and most importantly high variability in adsorption. Variability is of most concern with any drug affecting the clotting cascade, care is needed since complete blockage of the clotting cascade is an unwanted side effect. On the other hand low exposure levels to the compound will not lead to any therapeutic benefit. Therefore, good oral bioavailability is required and, particularly, low variability.

We have found with the polymers described above that they act as solubilising enhancers as well as precipitation inhibitors, also the polymers are self dispersing, water miscible and micelle forming.

We present as a feature of the invention an oral pharmaceutical composition comprising a compound and water miscible micelle forming block copolymer (hereinafter called “the copolymer”). Ideally the copolymer is a diblock copolymer of formula AB or BA. However the copolymer could also be a triblock copolymer of formula ABA or BAB. The copolymer could also be a multiblock copolymer having repeating BA or AB units of formula A(BA)n or B(AB)n, where n is an integer (preferably the copolymer is a diblock copolymer of formula AB or BA) and wherein

A is selected from a group consisting of

poly D-, L-, DL-lactic acid,

poly D-, L-, DL-lactide,

poly-glycolic acid,

polyglycolide,

polylactide-co-glycolide,

poly-ε-caprolactone, and

poly(3-hydroxybutyric); and

B is selected from a group of hydrophilic polymers consisting of

polyvinylalcohol,

polyvinylpyrrolidone,

polyethylene oxide, and

polyethylene glycol; or the hydrophilic polymer B may itself be a copolymer, for example a polyoxyethylene/polyoxypropylene block copolymer of the type known as Pluronics or synperonics.

A further feature of the invention is the use of water miscible micelle forming block copolymer in improving the oral bioavailability and/or variability of adsorption of a compound. Ideally the copolymer is a diblock copolymer of formula AB or BA. However the copolymer could also be a triblock copolymer of formula ABA or BAB. The copolymer could also be a multiblock copolymer having repeating BA or AB units of formula A(BA)n or B(AB)n, where n is an integer (preferably the copolymer is a diblock copolymer of formula AB or BA) and wherein

A is selected from a group consisting of

poly D-, L-, DL-lactic acid,

poly D-, L-, DL-lactide,

poly-glycolic acid,

polyglycolide,

polylactide-co-glycolide (PLGA),

poly-ε-caprolactone, and

poly(3-hydroxybutyric acid); and

B is selected from a group of hydrophilic polymers consisting of

polyvinylalcohol,

polyvinylpyrrolidone,

polyethylene oxide, and

polyethylene glycol; or the hydrophilic polymer B may itself be a copolymer, for example a polyoxyethylene/polyoxypropylene block copolymer of the type known as Pluronics or synperonics;

in improving the oral bioavailability and/or variability of adsorption of a compound.

The compound is an organic molecule of MW<800, the formulation working best with compounds which are poorly aqueous soluble and also with a compound which is basic, adsorbed after administration in the small intestine and in which such compound has significantly lower solubility in the pH conditions found at the site of adsorption than in the stomach.

Preferably the copolymer is a diblock copolymer of formula AB or BA or triblock copolymer of formula ABA or BAB. More preferably the copolymer is a diblock copolymer of formula AB or BA. Preferably the A block segment of the block copolymer, is a poly-(D,-L- or DL-lactic acid) or poly (D-,L- or DL-lactide). Preferably the Mw is between 500 Da and 5000 Da. More preferably between 1000 Da and 3000 Da and even more preferably between 1500 Da and 2000 Da. Preferably the B block segment of the copolymer is a polyethylene glycol, preferably methoxy-polyethylene glycol. Preferably the Mw is between 500 Da and 10,000 Daltons, more preferably between 1,000 Da and 5000 Da.

The most preferred copolymer is an AB diblock copolymer where A is a poly-(D-,L- or DL-lactic acid) or poly (D-,L- or DL-lactide) of Mw 2000 Da and B is a methoxypolyethylene glycol of Mw 2000Da.

The polymer may be judged to be micelle forming by a person skilled in the art by determination of the Critical Micelle Concentration (cmc). The formation of micelles of the copolymer in an aqueous environment is supported by the detection of the cmc, which can be measured using the Wilhelmy plate method. (S. A Hagan, A. G. A Coombes, M. C. Garnett, S. E. Dunn, M. C. Davies, L. Illum and S. S. Davis, Langmuir 1996, 12, 2153-2161)

Methods for the preparation of the polymers used are described in U.S. Pat. Nos. 4,942,035 and 4,526,938 or EPO,166,596, B1 Zhu. K. J, Lin. X. Z and Yang S. L. Preparation, characterisation and properties of polylactide (PLA)-poly(ethyleneglycol) (PEG) copolymers. J Appl. Polym.-Sci., 39(1990)

By the use of the term “significantly lower solubility in the pH conditions found at the site of adsorption than in the stomach” we mean that the solubility of the compound is at least 10x more soluble in the pH conditions found in the stomach (pH1-2) than the pH conditions found in the small intestine, (pH6-9), preferably 20x, 30x, 40x, 50x and X100

We have found in in vitro tests that the maximum supersaturated concentration of Compound 1 is improved by 4-10 times by use of the polymers described above.

A preferred ratio of copolymer to compound is from 10:1 to 0.25:1. Preferably 5:1 to 1:1

A preferred compound is Compound 1, 1-(5-chloroindol-2-ylsulfonyl)-4-[4-(4-pyridyl)benzoyl] piperazin (hereinafter called Compound 2) and 1-(5-chloroindol-2-ylsulfonyl)-4-[4-(1-imidazolyl)benzoyl] piperazine (hereinafter called Compound 3). Compound 2 and Compound 3 are disclosed in Examples 3 and 6 respectively of WO9957113. Compound 2 and 3 like Compound 1 are Factor Xa inhibitors.

The composition may contain from 0.01 mg to 1 g of compound. Additional excipients may be included in the composition.

Typically the compound will be present in an amount within the range of 1 to 80%, and preferably from 1 to 50% (especially 2 to 15% 2 to 20%) by weight of the composition.

The composition may be made by admixture of the compound and polymer, preferably by cryo-grinding the polymer and mixing with the compound, compression then may be used. Preferred methods for preparing a composition is as a solid dispersion, such techniques are known in the art and typically comprise the steps of dissolving the compound and the polymer in a common solvent and evaporating the solvent. Methods for evaporating the solvent include rotary evaporation, spray drying with appropriate excipients, lyophilization and thin film evaporation. Other techniques may be used such as solvent controlled precipitation, pH controlled precipitation, supercritical fluid technology and hot melt extrusion. To aid the process the melt may be extruded with any necessary additional excipient such as a plasticiser, including supercritical fluids. With hot melt extrusion the melt may be extruded or filled directly into capsules

When referring to a solid dispersion we do not exclude the possibility that a proportion of the compound may be dissolved within the polymer used, the exact proportion, if any, will depend upon the physical properties of the compound and the polymer selected.

Conventional excipients which may be added include preservatives, stabilisers, antioxidants, silica flow conditioners, antiadherents or glidants.

The invention is illustrated below by the following non-limiting examples.

Preparation of Solid Dispersion

For a 1:5 Ratio

0.5 g of drug (Compound 1) and 2.5 g of polymer are weighed directly into a 250 ml round bottom flask and dissolved in 63 ml of methanol/dichloromethane (50:50). The solvent was removed on the rotary evaporator. The formulation was placed in a vacuum oven and dried under high vacuum at 40° C. for 48 hours.

Weights and volumes for other ratio's are pro-rata to the above formulation.

Solubility Measurements

| Solubility | Compound 1 | |
|---|---|---|
| Water | <5 | ug/ml |
| pH 1.2 | 250 | ug/ml |
| pH 6.8 | 2 | ug/ml |

In Vitro Dissolution of Solid Dispersions

pH Shift Dissolution Method

The formulations were weighed into hard gelatin capsules (equivalent to 25 mg drug) and dissoluted in 500 ml 0.1N HCl for one hour at 37° C. (paddle speed 100 rpm). A 5 ml sample was taken at 55 minutes and the media replaced. After one hour 10 ml of a 2.5M KH₂PO₄/16.72% (w/v) NaOH solution was added to the HCl to shift the pH to 6.5. 5 ml samples were then removed with a plastic syringe at 5, 15, 30, 45 and 60 minutes and media replaced after every sampling time point. Each sample was centrifuged (14,000 rpm) at ambient temperature for 15 minutes and then analysed by HPLC using the following conditions:

| | |
|---|---|
| Eluent: | 40% ACN/60% water/0.2% TFA |
| column: | 25 cm HIRPB 4.6 mm i.d..(with guard) |
| detection wavelength: | 236 nm |
| flow rate: | 1.5 ml/min |
| temperature: | ambient |
| injection volume: | 80 μl |
| retention time: | approximately 6 minutes |

pH 6.5 Dissolution Method

The formulations were weighed into hard gelatin capsules (equivalent to 25 mg drug) and dissoluted in media comprising of 500 ml 0.1N HCl and 10ml of a 2.5M KH₂PO₄/16.72% (w/v) NaOH solution for one hour at 37° C. (paddle speed 100 rpm). 5 ml samples were then removed with-a-plastic syringe at 5, 10, 20, 30, 45 and 60 minutes and media replaced after every sampling time point. Each sample was centrifuged (14,000 rpm) at ambient temperature for 15 minutes and then analysed by HPLC using the same conditions as the pH shift method.

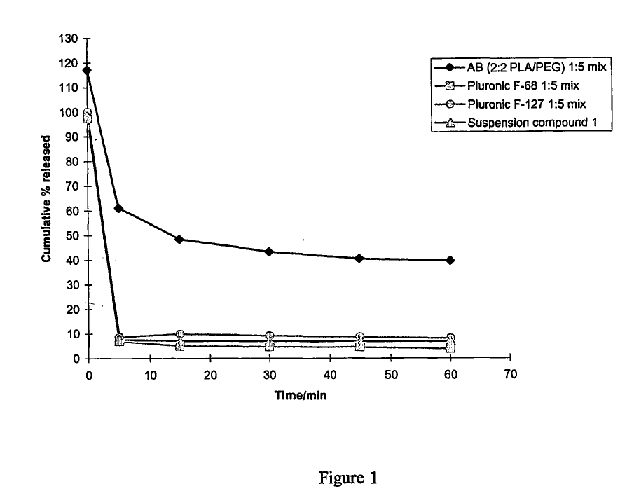

FIG. 1 shows the release profile of a solid dispersion of Compound 1 with a PLA:PEG AB block copolymer and Pluronic polymers using the pH shift dissolution method. A conventional suspension of Compound 1 was included for comparison. This figure demonstrates that the PLA:PEG polymer is the optimal solid dispersion matrix material since the highest levels of supersaturation are attained with this polymer. The solid dispersions made with Pluronic F-68 and F-127 do not provide any great advantage over a conventional suspension of Compound 1. Similarly to the conventional suspension, on shifting to the higher pH, the Pluronic formulations are not capable of maintaining supersaturated levels.

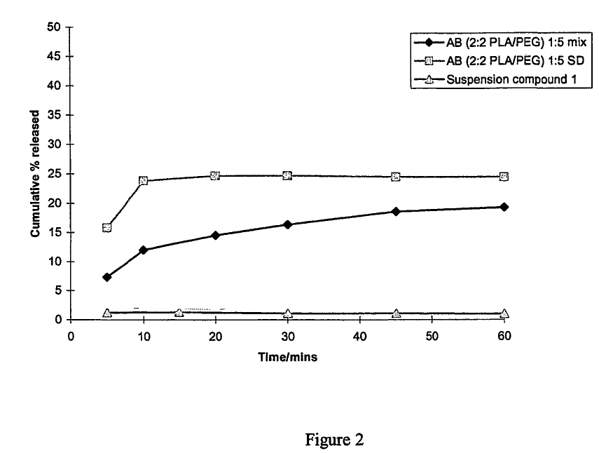

FIG. 2 shows the release profile of two PLA:PEG AB block copolymer formulations of Compound 1 (SD is a solid dispersion and mix is an admixture) in the pH 6.5 dissolution test. A conventional suspension of Compound 1 was included for comparison. This figure demonstrates that in the absence of any prior formulation, the PLA:PEG polymer is capable of enhancing the dissolution of Compound 1 (admixture). This may be as a result of the polymer solubilising the compound.

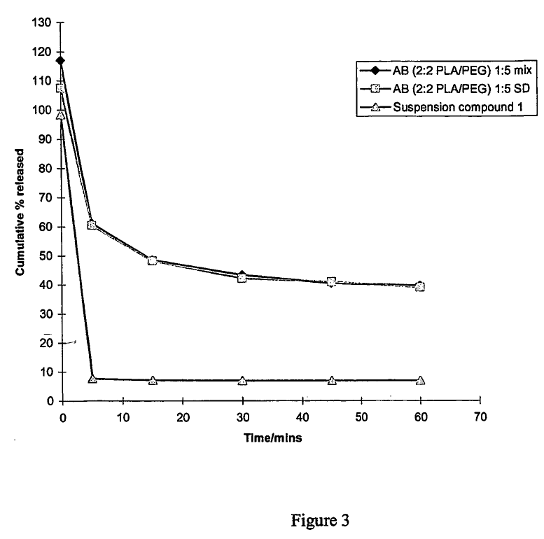

FIG. 3 shows the release profile of two PLA:PEG AB block copolymer formulations of Compound 1 (SD is a solid dispersion and mix is an admixture) in the pH shift dissolution test. A conventional suspension of Compound 1 was included for comparison. This figure demonstrates that the PLA:PEG polymer is capable of maintaining supersaturated levels of the compound 1 in both the formulated and non-formulated state (i.e. SD or mix). FIGS. 2 and 3 demonstrate that the PLA:PEG's could be acting by a combination of solubilisation and inhibition of precipitation.

1. An oral pharmaceutical composition comprising a compound and a diblock copolymer of formula AB or BA or a triblock copolymer of formula ABA or BAB or a multiblock copolymer having repeating BA or AB units of formula A(BA)n or B(AB)n, where n is an integer and wherein
A is selected from a group consisting of
poly D-, L-, DL-lactic acid,
poly D-, L-, DL-lactide,
poly-glycolic acid,
polyglycolide,
polylactide-co-glycolide,
poly-ε-caprolactone, and
poly(3-hydroxybutyric acid); and
B is selected from a group of hydrophilic polymers consisting of
polyvinylalcohol,
polyvinylpyrrolidone,
polyethylene oxide, and
polyethylene glycol; or the hydrophilic polymer b may itself be a copolymer, for example a
polyoxyethylene/polyoxypropylene block copolymer of the type known as Pluronics or synperonics:

2. Use of a water miscible micelle forming diblock copolymer of formula AB or BA or a triblock copolymer of formula ABA or BAB or a multiblock copolymer having repeating BA or AB units of formula A(BA)n or B(AB)n, where n is an integer and wherein
A is selected from a group consisting of
poly D-, L-, DL-lactic acid,
poly D-, L-, DL-lactide,
poly-glycolic acid,
polyglycolide,
polylactide-co-glycolide (PLGA),
poly-ε-caprolactone, and
poly(3-hydroxybutyric acid); and
B is selected from a group, of hydrophilic polymers consisting of
polyvinylalcohol,
polyvinylpyrrolidone,
polyethylene oxide, and
polyethylene glycol; or the hydrophilic polymer B may itself be a copolymer, for example a
polyoxyethylene/polyoxypropylene block copolymer of the type known as Pluronics or synperonics;
in improving the oral bioavailability and/or variability of adsorption of a compound.

3. An oral pharmaceutical composition as claimed in claim 1 or use of a water miscible micelle forming copolymer as claimed in claim 2 wherein the A block segment of the copolymer is a poly-(D-,L- or DL-lactic acid) or poly (D-,L- or DL-lactide).

4. An oral pharmaceutical composition as claimed in claim 3 or use of a water miscible micelle forming copolymer as claimed in claim 3 wherein the Mw of the A polymer is between 500 Da and 5,000 Da.

5. An oral pharmaceutical composition as claimed in claim 4 or use of a water miscible micelle forming copolymer as claimed in claim 4 wherein the Mw of the A polymer is between 1000 Da and 3000 Da.

6. An oral pharmaceutical composition as claimed in claim 5 or use of a water miscible micelle forming copolymer as claimed in claim 5 wherein the Mw of the A polymer is between 1300 Da and 2200 Da.

7. An oral pharmaceutical composition as claimed in claim 6 or use of a water miscible micelle forming copolymer as claimed in claim 6 wherein the Mw of the A polymer is 2000 Da.

8. An oral pharmaceutical composition as claimed in any one of claims 1 or 3 to 7 or use of a water miscible micelle forming copolymer as claimed in any claim from 2 to 7 wherein the B block segment of the copolymer is a polyethylene glycol.

9. An oral pharmaceutical composition as claimed in claim 8 or use of a water miscible micelle forming copolymer as claimed in claim 8 wherein the B block segment of the copolymer is methoxy-polyethylene glycol.

10. An oral pharmaceutical composition as claimed in claim 8 or 9 or use of a water miscible micelle forming copolymer as claimed in claim 8 or 9 wherein the Mw of the B polymer is between 500 Da and 10,000 Da.

11. An oral pharmaceutical composition as claimed in claim 10 or use of a water miscible micelle forming copolymer as claimed in claim 10 wherein the Mw of the B polymer is between 1,000 Da and 5000 Da.

12. An oral pharmaceutical composition as claimed in any one of claims 1, or 3 to 11 or use of a water miscible micelle forming copolymer as claimed in any one of claims 2 to 11 wherein the copolymer is a diblock copolymer of formula AB or BA.

13. An oral pharmaceutical composition as claimed in any one of claims 1 or 3 to 11 or use of a water miscible micelle forming copolymer as claimed in any one of claims 2 to 11 wherein the copolymer is a triblock copolymer of formula ABA or BAB.

14. An oral pharmaceutical composition comprising a compound and a diblock copolymer of formula AB or BA wherein A is a polyL-lactide of Mw of 2000 Da and B is a polyethylene glycol of Mw of 2000 Da.

15. An oral pharmaceutical composition comprising a compound and a diblock copolymer of formula AB or BA wherein A is a poly-(D-,L- or DL-lactic acid) or poly (D-,L- or DL-lactide) of Mw 2000 Da and B is a methoxypolyethylene glycol of Mw 2000Da.

16. An oral pharmaceutical composition as claimed in any one of claims 1 or 3 to 15 wherein the compound is selected from 1-(6-chloronaphth-2-ylsulfonyl)-4-[4-(4-pyridyl)benzoyl] piperazine, 1-(5-chloroindol-2-ylsulfonyl)-4-[4-(4-pyridyl)benzoyl] piperazine and 1-(5-chloroindol-2-ylsulfonyl)-4-[4-(1-imidazolyl)benzoyl] piperazine.

17. An oral pharmaceutical composition as claimed in any one of claims 1 or 3 to 15 wherein the ratio of copolymer to compound is from 10:1 to 0.25:1.

18. An oral pharmaceutical composition as claimed in any one of claims 1 or 3 to 15 wherein the composition comprises from 0.01 mg to 1 mg of compound.
